(19) United States
(12) Patent Application Publication
(10) Pub. No.: US 2022/0296919 A1
(43) Pub. Date: Sep. 22, 2022

(54) METHOD FOR TARGETED TREATING DERMATOSES

(71) Applicant: STRATA SKIN SCIENCES INC., Horsham, PA (US)

(72) Inventor: Dolev RAFAELI, Horsham, PA (US)

(21) Appl. No.: 17/640,125

(22) PCT Filed: Sep. 16, 2020
(86) PCT No.: PCT/IL2020/051017

§ 371 Date: Mar. 3, 2022

Related U.S. Application Data
(60) Provisional application No. 62/902,487, filed on Sep. 19, 2019.

(51) Int. Cl. A61N 5/06; A61N 5/067; A61P 17/06; A61K 9/00

(52) U.S. Cl. CPC A61N 5/0616; A61N 5/067; A61P 17/06; A61K 9/0014; A61N 2005/0628; A61N 2005/0661; A61N 2005/0644

(57) ABSTRACT

Disclosed are methods for localized treatment of a skin condition including administering a therapeutically effective amount of at least one biological drug to the subject, utilizing a dosimetry device to transmit varying percentages of the UVB light to an area of the subject's skin; assessing a response of the treated area to the varying percentages of the UVB light transmitted thereto; and determining an optimal dose of UVB light, based on the response of the treated area to the varying percentages of UVB light and to the biological drug; and applying the optimal dose of UVB light to the treatment area.

### FIELD OF TECHNOLOGY

The present application generally relates to methods of treating dermatoses, and, more specifically, to optimizing phototherapy treatment protocols in view of other medications, in particular biologic agents, administered to the subject in conjunction with the phototherapy.

### BACKGROUND

Psoriasis, vitiligo and other skin conditions affect millions of people. These dermatoses can range from mild to severe and can lead to substantial morbidity and psychological stress, and can have a profound negative impact on the quality of life of an individual suffering from a skin condition. Although available therapies can reduce the extent and severity of these diseases and improve an individual's quality of life, reports have indicated dissatisfaction with the effectiveness, cost, and inconvenience of current treatment modalities.

Dermatoses such as psoriasis can range in severity from relatively mild, with some drying and flaking of the affected skin, to severe cases with very severe outbreaks over large areas of the patient's body. Approximately one-third of patients experience moderate to severe psoriasis. Even very mild psoriasis is uncomfortable and unsightly. Severe cases can be physically and psychologically debilitating, presenting a very serious threat to the patient's overall health.

Psoriasis can be divided into various types according to the affected area and/or symptoms. For example, plaque psoriasis (e.g., psoriasis vulgaris) is a common form of the condition and accounts for about 80% to about 90% of patients. Plaque psoriasis typically appears as red patches or plaques with dry, silvery scales. Another type is guttate psoriasis, which is characterized by numerous small round spots. Guttate psoriasis often renders these numerous round spots in large areas of the body, such as the trunk, limbs, and scalp. Flexural psoriasis (inverse psoriasis), on the other hand, appears as smooth inflamed patches of skin. Flexural psoriasis occurs in skin folds such as areas around the genitals, the armpits, the overweight stomach, and the breasts. Pustular psoriasis appears as raised bumps and is commonly found locally in the hands and feet, but it can extend to other parts of the body. Erythrodermic psoriasis usually comes with severe itching, swelling, and pain. These radical symptoms may involve the widespread inflammation and exfoliation of the skin. Fingernails and toenails may be affected by nail psoriasis, and often undergo a variety of changes in the appearance of the nail. Small indentations in the nails (e.g., pitting), lifting up of the nails, discoloration, thickening, and crumbling of nails may appear due to nail psoriasis. Certain embodiments disclosed herein may be used to treat any type or combination of types of psoriasis, some of which are described above. In certain embodiments, the methods described herein may be used to treat one specific type of psoriasis. In certain alternative embodiments, the methods described herein may be used to treat two or more types of psoriasis.

The severity of psoriasis can be classified or “scored” in a variety of ways. This disease varies from causing relatively minor plaques in a localized area of the body to a generalized psoriasis covering a substantially large area of the body. In a classification method that is based on the surface area of tissue affected, psoriasis can be graded as mild (e.g., affecting less than about 3% of the total area of the body surface (BSA)), moderate (e.g., affecting about 3% to about 10% BSA), or severe (e.g., affecting more than about 10% BSA). By way of comparison, the palm of a person's hand is about 1% BSA. Other scales may also be employed for measuring the severity of psoriasis. For example, in addition to the size of affected or influenced BSA, factors such as the condition's duration, the frequency of disease recurrence, disease activity (e.g., degree of plaque redness, thickness, and scaling), response to previous therapies, and the impact of the disease on the person may also be considered to determine the severity of the disease. Therefore, psoriasis may be characterized as severe if at least one of the following is observed: the area of influenced tissue is greater than about 10% BSA; the condition (e.g., accompanied by pain and/or swelling) persists for a month or more; the disease activity is substantially active; and the disease is resistant to one or more of known treatments.

Severity of psoriasis may be determined according to standard clinical definitions. For example, the Psoriasis Area and Severity Index (PAST) assesses psoriasis disease intensity based on the quantitative assessment of three typical signs of psoriatic lesions: erythema, infiltration, and desquamation, combined with the skin surface area involvement in the four main body areas: head, trunk, upper extremities, and lower extremities. Since its development in 1978, PASI has been used throughout the world by clinical investigators. PASI scores range from 0 (no disease) to 72 (maximum disease), in which higher scores indicate greater disease severity. Improvements in psoriasis are indicated as “PASI 50” (a 50% improvement in PASI from baseline), “PASI 75” (a 75% improvement in PASI from baseline), “PASI 90” (a 90% improvement in PASI from baseline), “PASI 95” (a 95% improvement in PASI from baseline), and “PASI 100” (a 100% improvement in PASI from baseline).

The Physicians Global Assessment (PGA) also assesses psoriasis activity and clinical response to treatment. PGA is a six-point score that summarizes the overall quality (erythema, scaling, and thickness) and extent of plaques relative to the baseline assessment. A patient's response is rated as worse (negative clearance (disease became worse)), poor (0-24% clearance), fair (25-49% clearance), good (50-74% clearance), excellent (75-99% clearance), or cleared (100% clearance).

In normal skin, varying shades of brown are seen (depending on a person's race) representing the pigment melanin. This pigment is produced by a cell type known as a melanocyte. In vitiligo, there is an absence of melanocytes in the areas afflicted with the disorder. This loss of pigment results in the affected areas being completely white. This condition has a predilection for the skin around the mouth and the eyes. The result is cosmetically disfiguring, especially for dark skinned people. Furthermore, the depigmented skin is sun sensitive, and thus is subject to sunburns and skin cancer. In sum, vitiligo is both cosmetically and practically distressing to patients afflicted with the disease.

Methods and apparatuses for targeted phototherapy (e.g., narrow-band, 308 nm excimer lasers dispensing ultraviolet light energy) are known as an effective and safe treatment for various dermatoses (e.g., psoriasis, vitiligo, leukoderma, atopic dermatitis, and alopecia areata).

With conventional UVB phototherapy, dosing is predicated on either an individual's Fitzpatrick Skin Type (i.e., skin color and darkness) in conjunction with the thickness of the psoriatic plaque or on a measurement of an individual's minimum erythemal dose (MED). An individual's minimum erythemal dose is the dose of UVB that generates a significant red erythemal skin response in normal/healthy tissue. However, neither of these two methods of determining an individual's appropriate dosing protocol is therapeutically optimal and typically results in dosing at levels that are far too conservative which, in turn, results in a reduced therapeutic benefit. This is because using the Fitzpatrick Skin Type is merely a guess at an individual's maximum tolerable dose (MTD) (based on historical norms that do not apply to many individuals) and the fundamental limitations of the minimum erythemal dose method that only measures the tolerance of the healthy/normal tissue, not the diseased tissue being treated. In either case, many individuals are regularly administered sub-optimal UVB dosing when clinicians, recognizing that current dosing paradigms are only a crude guess, initiate dosing at even lower levels than might be expected. They do so to avoid unintentional dosing at higher levels than the individual's minimal blistering dose (MBD) leading to extreme erythema, blistering, and possible injury. This problem is enhanced by the fact that the optimum dose (OTD) can vary greatly for each individual as well as in between plaques of a same individual, making it very difficult, if not impossible, to correctly gauge an individual's optimal dose. The variability is further augmented when the phototherapy is administered in conjunction with other medicaments which may likewise influence the effectivity and/or sensitivity to the treatment.

As such, the lack of having an objective means of determining an individual's minimal blistering dose prevents clinicians from dosing more effectively at an individual's optimum dose level, which could significantly lower the total number of required UVB treatment sessions to obtain the desired clinical outcome.

As a result of the typically high number of treatment sessions required, the use of phototherapy is commonly limited due to the overall inconvenience of the therapy. Poor compliance with the necessary regimen of regular treatment sessions is common because of the time, travel and cost, in many cases, to effectively treat the disease. Other less effective therapies (e.g., topical prescriptions and over-the-counter topical creams) are often an individual's more convenient fallback option.

Biologic agents (also referred to herein as “biologics”) have gained popularity in treating severe psoriasis. Examples of injectable biologics include: alefacept (e.g., Amevive®, available from Biogen, Inc. of Cambridge, Mass.); etanercept (e.g., Enbrel®, available from Immunex Corporation of Seattle, Wash.); adalimumab (e.g., Humira®, available from Abbott Laboratories of Abbott Park, Ill.); infliximab (e.g., Remicade®, available from Centocor, Inc. of Malvern, Pa.); and ustekinumab (e.g., Stelara®, available from Johnson & Johnson of New Brunswick, N.J.).

These biologic medications, while effective in many patients, work by inducing systemic immunosuppression with increased risk of malignancies, infections including tuberculosis and histoplasmosis, congestive heart failure, lupus-like syndrome, demyelinating diseases, etc. In addition to side effects from systemic immunosuppression, biologics can have major organ toxicity as a potential side effect including bone marrow suppression, liver toxicity, kidney toxicity, hypertension, teratogenicity, etc. Moreover, these biologics can have severe adverse effects such as nausea, fatigue, difficulty sleeping, vomiting, headaches, easy bruising and bleeding, fever, diarrhea, and chills and the side-effects of biologic treatment often become more severe or likely with prolonged use.

In addition, biologics can be very expensive, for example reported to be about $20,000-$70,000 per year.

### SUMMARY

Aspects of the disclosure, in some embodiments thereof, relate to methods for providing an optimized phototherapy treatment to a subject's skin area affected with a skin condition, wherein the phototherapy is provided in conjunction with an additional treatment regimen, preferably wherein the additional treatment regimen is administration of a biologic.

According to some embodiments, the biological may be selected from the group of: alefacept, etanercept, adalimumab, infliximab, ustekinumab and any combination thereof. Each possibility is a separate embodiment.

It was found by the inventors of the present invention that the effectiveness and/or the sensitivity of a phototherapy treatment may be altered when a drug or other therapy is provided in conjunction with the phototherapy.

For example, phototherapy may cause inflammation, particularly as the phototherapy dosages increase, and, as a result, reduce the maximum dose typically provided to the patient. If, however, the phototherapy is administered in conjunction with therapeutics having anti-inflammatory effects (e.g. alefacept), the maximum tolerated dose may be increased, and a faster and/or more efficient overall therapy may thus be ensured. That is, patients sensitive to the UVB treatment (i.e. patients with a relatively low maximum tolerated dose) may acquire a higher maximum tolerated dose due to the combined treatment of UVB with alefacept.

Moreover, certain drugs may absorb and/or interfere with the phototherapy, thereby increasing the dose required to obtain a desired outcome. On an opposite hand, some medicaments may increase the sensitivity and/or efficiency of phototherapy, for example by causing a thinning of the skin, which in turn increases the penetrability of the UVB light. Accordingly, administration of a drug in conjunction with UVB treatment may reduce the optimal and or maximum tolerated dose of UVB light transmitted to a skin area.

Such influences of medicaments on the efficacy of phototherapy may make it even more difficult to establish the optimum dosage of a phototherapy treatment.

Advantageously, the hereindisclosed method enables efficient determining of an optimal dose of UVB light that should be provided to a subject in need thereof. By utilizing a dosimetry device including an optical matrix with a plurality of regions configured to allow varying percentages of UVB light to pass therethrough, the method enables transmitting varying percentages of UVB light to a treatment area, following which a response to the treatment (e.g. degree of blistering in the treated area). Based upon the response to the UVB light transmission and the biologics administered, the optimal dose of UVB light and/or of the biologic may be determined.

Moreover, certain medicaments should, due to their side effects, only be administered for a short period of time and/or at a lowest possible concentration.

Advantageously, the efficient determining of a maximal tolerable dose of UVB light may shorten the duration and/or concentration of the medicament (e.g. alefacept) administered to the subject. As a non-limiting example, patients more tolerant to the UVB light treatment may need a lower dose of the biologic to gain optimal treatment results. As another, non-limiting example, the dose of UVB light, provided to a patient, is typically increased from session to session, due to acquired tolerance (desensitization) to the treatment, and the amount of the biologic may therefore optionally be reduced accordingly.

According to some embodiments, there is provided a method for localized treatment of a skin condition, the method comprising the steps of administering a therapeutically effective amount of at least one biological drug to the subject, utilizing a dosimetry device, comprising an optical matrix comprising a plurality of regions, each region configured to allow varying percentages of UVB light to pass therethrough, to transmit varying percentages of the UVB light to an area of the subject's skin; assessing a response of the treated area to the varying percentages of the UVB light transmitted thereto; determine an optimal dose of UVB light, based on the response of the treated area to the varying percentages of UVB light and the biological drug administered; and applying the optimal dose of UVB light to the treatment area.

According to some embodiments, the optimal dose of UVB light is the maximum tolerable dose of UVB light.

According to some embodiments, the method further comprises determining an optimal amount of the biological drug, based on the determined maximum tolerable dose of UVB light.

According to some embodiments, the transmission of light passing through the regions ranges from about 20% in one region up to about 100% in another region. According to some embodiments, the transmission of light passing through the regions ranges from about 0% in one region up to about 90% in another region.

According to some embodiments, the UVB light is UVB laser light having a wavelength of about 290-320 nm. According to some embodiments, the UVB light is UVB laser light having a wavelength of about 308 nm. According to some embodiments, the UVB laser light has an intensity of 60 mwatts.

According to some embodiments, the treatment area is assessed approximately 24 to 48 hours after the varying percentages of the UVB light are applied thereto.

According to some embodiments, the administering of the biological drug and the applying of the maximum tolerable dose of UVB light is repeated 1-5 times a week.

According to some embodiments, the assessing of the response of the treatment area to the varying percentages of the UVB light transmitted utilizing the dosimetry device is repeated at least every two weeks. According to some embodiments, the method further comprises adjusting the maximum tolerable dose of UVB light, based on the repeated assessment of the response of the treatment area to the varying percentages of the UVB light. According to some embodiments, the method further comprises adjusting the therapeutically effective amount of the at least one biological drug, based on the repeated assessment of the response of the treatment area to the varying percentages of the UVB light.

According to some embodiments, the administering of the therapeutically effective amount of the at least one biological drug is initiated at least 2 days prior to the transmitting of the varying percentages of UVB light to a treatment area and the assessment of the response of the treated area to the varying percentages of the UVB light transmitted thereto.

According to some embodiments, the at least one biological drug is selected from the group consisting of: alefacept, etanercept, adalimumab, infliximab, ustekinumab and any combination thereof.

According to some embodiments, the skin condition is selected from vitiligo, psoriasis, leukoderma, atopic dermatitis, dyshidrosis, eczema, alopecia areata and lichen planus. According to some embodiments, the skin condition is vitiligo. According to some embodiments, the skin condition is psoriasis.

According to some embodiments, there is provided a method for localized treatment of a skin condition, the method comprising the steps of utilizing a dosimetry device, comprising an optical matrix comprising a plurality of regions, each region configured to allow varying percentages of UVB light to pass therethrough, to transmit varying percentages of the UVB light to an area of the subject's skin affected with the skin condition; assessing a response of the skin area to the varying percentages of the UVB light transmitted thereto; determining an optimal dose of UVB light, based on the response of the treated skin area to the varying percentages of UVB light; determining an optimal dose of at least one biological drug, based on the determined optimal dose of UVB light; administering the optimal dose of the biological drug to the subject, and treating the skin area with the optimal dose of UVB light.

According to some embodiments, the optimal dose of UVB light is the maximum tolerable dose of UVB light.

According to some embodiments, the transmission of light passing through the regions ranges from about 20% in one region up to about 100% in another region. According to some embodiments, the transmission of light passing through the regions ranges from about 0% in one region up to about 90% in another region.

According to some embodiments, the UVB light is UVB laser light having a wavelength of about 290-320 nm. According to some embodiments, the UVB light is UVB laser light having a wavelength of about 308 nm. According to some embodiments, the UVB laser light has an intensity of 60 mwatts.

According to some embodiments, the treatment area is assessed approximately 24 to 48 hours after the varying percentages of the UVB light are applied thereto.

According to some embodiments, the administering of the biological drug and the applying of the maximum tolerable dose of UVB light is repeated 1-5 times a week.

According to some embodiments, the assessing of the response of the treatment area to the varying percentages of the UVB light transmitted utilizing the dosimetry device is repeated at least every two weeks. According to some embodiments, the method further comprises adjusting the maximum tolerable dose of UVB light, based on the repeated assessment of the response of the treatment area to the varying percentages of the UVB light. According to some embodiments, the method further comprises adjusting the therapeutically effective amount of at least one biological drug, based on the repeated assessment of the response of the treatment area to the varying percentages of the UVB light.

According to some embodiments, the at least one biological drug is selected from the group consisting of: alefacept, etanercept, adalimumab, infliximab, ustekinumab and any combination thereof.

According to some embodiments, the skin condition is selected from vitiligo, psoriasis, leukoderma, atopic dermatitis, dyshidrosis, eczema, alopecia areata and lichen planus. According to some embodiments, the skin condition is vitiligo. According to some embodiments, the skin condition is psoriasis.

According to some embodiments, there is provided a method for determining an optimal treatment protocol for localized treatment of a skin condition, the method comprising analyzing data regarding a response of a skin area of a subject treated with varying percentages of the UVB light, and with at least one biological drug, and determining an optimal dose of UVB light, based on the analyzed response of the treated area to the varying percentages of UVB light and to the biological drug.

According to some embodiments, the varying percentages of UVB are transmitted to the skin area utilizing a dosimetry device, comprising an optical matrix comprising a plurality of regions, each region configured to allow varying percentages of UVB light to pass therethrough. According to some embodiments, the transmission of light passing through the regions ranges from about 20% in one region up to about 100% in another region. According to some embodiments, the transmission of light passing through the regions ranges from about 0% in one region up to about 90% in another region.

According to some embodiments, the UVB light is UVB laser light having a wavelength of about 290-320 nm. According to some embodiments, the UVB light is UVB laser light having a wavelength of about 308 nm.

According to some embodiments, the UVB laser light has an intensity of 60 mwatts.

According to some embodiments, the optimal dose of UVB light is the maximum tolerable dose of UVB light. According to some embodiments, the method further comprises determining an optimal amount of the biological drug, based on the determined maximum tolerable dose of UVB light.

According to some embodiments, the treatment area is assessed approximately 24 to 48 hours after the varying percentages of the UVB light were applied thereto.

According to some embodiments, the assessing of the response of the treatment area to the varying percentages of the UVB light transmitted utilizing the dosimetry device is repeated at least every two weeks. According to some embodiments, the method further comprises adjusting the maximum tolerable dose of UVB light, based on the repeated assessment of the response of the treatment area to the varying percentages of the UVB light.

According to some embodiments, the method further comprises adjusting the therapeutically effective amount of at least one biological drug, based on the repeated assessment of the response of the treatment area to the varying percentages of the UVB light.

According to some embodiments, the administering of the therapeutically effective amount of the at least one biological drug was initiated at least 1 week prior to the transmitting of the varying percentages of UVB light to a treatment area and/or prior to the assessment of the response of the treated area to the varying percentages of the UVB light transmitted thereto.

According to some embodiments, the at least one biological drug is selected from the group consisting of is selected from the group consisting of: alefacept, etanercept, adalimumab, infliximab, ustekinumab and any combination thereof. Each possibility is a separate embodiment.

According to some embodiments, the skin condition is selected from vitiligo, psoriasis, leukoderma, atopic dermatitis, dyshidrosis, eczema, alopecia areata and lichen planus. Each possibility is a separate embodiment. According to some embodiments, the skin condition is vitiligo. According to some embodiments, the skin condition is psoriasis.

According to some embodiments, there is provided a method for determining an optimal treatment protocol for localized treatment of a skin condition, the method comprising analyzing data regarding a response of a skin area of a subject treated with varying percentages of the UVB light, determining an optimal dose of UVB light, based on the analyzed response of the treated area to the varying percentages of UVB light, and determining an optimal dose of at least one biological drug to be administered based on the determined optimal dose.

According to some embodiments, the varying percentages of UVB are transmitted to the skin area utilizing a dosimetry device, comprising an optical matrix comprising a plurality of regions, each region configured to allow varying percentages of UVB light to pass therethrough. According to some embodiments, the transmission of light passing through the regions ranges from about 20% in one region up to about 100% in another region. According to some embodiments, the transmission of light passing through the regions ranges from about 0% in one region up to about 90% in another region.

According to some embodiments, the UVB light is UVB laser light having a wavelength of about 290-320 nm. According to some embodiments, the UVB light is UVB laser light having a wavelength of about 308 nm.

According to some embodiments, the UVB laser light has an intensity of 60 mwatts.

According to some embodiments, the optimal dose of UVB light is the maximum tolerable dose of UVB light. According to some embodiments, the method further comprises determining an optimal amount of the biological drug, based on the determined maximum tolerable dose of UVB light.

According to some embodiments, the treatment area is assessed approximately 24 to 48 hours after the varying percentages of the UVB light were applied thereto.

According to some embodiments, the assessing of the response of the treatment area to the varying percentages of the UVB light transmitted utilizing the dosimetry device is repeated at least every two weeks. According to some embodiments, the method further comprises adjusting the maximum tolerable dose of UVB light, based on the repeated assessment of the response of the treatment area to the varying percentages of the UVB light.

According to some embodiments, the method further comprises adjusting the therapeutically effective amount of at least one biological drug, based on the repeated assessment of the response of the treatment area to the varying percentages of the UVB light.

According to some embodiments, the at least one biological drug is selected from the group consisting of alefacept, etanercept, adalimumab, infliximab, ustekinumab and any combination thereof. Each possibility is a separate embodiment.

According to some embodiments, the skin condition is selected from vitiligo, psoriasis, leukoderma, atopic dermatitis, dyshidrosis, eczema, alopecia areata and lichen planus. Each possibility is a separate embodiment. According to some embodiments, the skin condition is vitiligo. According to some embodiments, the skin condition is psoriasis.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

### BRIEF DESCRIPTION OF THE DRAWINGS

Some embodiments of the disclosure are described herein with reference to the accompanying figures. The description, together with the figures, makes apparent to a person having ordinary skill in the art how some embodiments of the disclosure may be practiced. The figures are for the purpose of illustrative discussion and no attempt is made to show structural details of an embodiment in more detail than is necessary for a fundamental understanding of the teachings of the disclosure. For the sake of clarity, some objects depicted in the figures are not to scale.

FIG. 1 is a perspective view of the hand-held phototherapy delivery apparatus and an embodiment of an end piece with a circular diaphragm connected thereto for beam shaping;

FIG. 2A is a front view of an embodiment of the dosimetry device of the present invention illustrating an embodiment of the photosensitivity matrix;

FIG. 2B is an end view of the matrix of FIG. 2A;

FIG. 3 is a flowchart of a method for localized treatment of a skin condition; according to some embodiments;

FIG. 4 is a flowchart of a method for treatment of a skin condition; according to some embodiments;

FIG. 5 is a flowchart of a method for treatment of a skin condition; according to some embodiments;

FIG. 6 is a flowchart of a method for treatment of a skin condition; according to some embodiments;

FIG. 7 is a flowchart of a method for treatment of a skin condition; according to some embodiments; and

FIG. 8 is a flowchart of a method for treatment of a skin condition; according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

Unless defined otherwise, all technical and scientific terms used herein have the same meaning as commonly understood by one of ordinary skill in the art to which the invention pertains.

The articles “a” and “an” are used herein to refer to one or to more than one (i.e. to at least one) of the grammatical object of the article. By way of example, “an element” means one element or more than one element.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein “biologic”, “biological agent”, “biological drug” and “biopharmaceutical” may be used interchangeably and refer to any pharmaceutical drug product manufactured in, extracted from, or semi-synthesized from biological sources. Non-limiting examples of biological drugs include antibodies, interleukins, interferons, peptides and proteins.

According to some embodiments, the biological drug may be selected from the group of: alefacept, etanercept, adalimumab, infliximab, ustekinumab and any combination thereof. Each possibility is a separate embodiment.

As used herein, term “adverse effect” includes, but is not limited to gastrointestinal, renal and hepatic toxicities, leukopenia, increases in bleeding times due to, e.g., thrombocytopenia, and prolongation of gestation, nausea, vomiting, somnolence, asthenia, dizziness, teratogenicity, extra-pyramidal symptoms, akathisia, cardiotoxicity including cardiovascular disturbances, inflammation, male sexual dysfunction, and elevated serum liver enzyme levels.

As used herein, the term “patient” refers to a mammal, particularly a human. In some embodiments, the patient is a female. In further embodiments, the patient is a male. In further embodiments, the patient is a child.

As used herein, and unless otherwise specified, the terms “treat,” “treating” and “treatment” contemplate an action that occurs while a patient is suffering from the specified disease or disorder, which reduces the severity or symptoms of the disease or disorder, or retards or slows the progression or symptoms of the disease or disorder.

As used herein, the term “pharmaceutical composition” or “composition” refers to a mixture of at least one compound and/or composition useful within the invention with a pharmaceutically acceptable carrier. The pharmaceutical composition facilitates administration of the compound and/or composition to a subject.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound and/or composition useful within the invention within or to the patient such that it may perform its intended function. Each carrier must be “acceptable” in the sense of being compatible with the other ingredients of the formulation, including the compound and/or composition useful within the invention, and not injurious to the patient.

The terms “pharmaceutically effective amount” and “effective amount” refer to a nontoxic but sufficient amount of an agent to provide the desired biological result. That result can be reduction and/or alleviation of the signs, symptoms, or causes of a disease or disorder, or any other desired alteration of a biological system. An appropriate effective amount in any individual case may be determined by one of ordinary skill in the art using routine experimentation.

The term “phototherapy” as used herein refers to controlled and/or prescribed application of light from an artificial light source to an area of a patient's skin in order to derive a therapeutic benefit.

As used herein, the terms “ultraviolet light” or “UV” refers to light with a wavelength between 10 and 400 nm, including, but not limited to, ultraviolet B (UVB, 280-320 nm) and ultraviolet A (UVA, 320-400 nm) and narrow regions thereof, e.g., narrowband ultraviolet B (nbUVB, 311-312 nm) and UVA1 (340-400 nm).

According to some embodiments, the biological medication may be selected from the group of: alefacept, etanercept, adalimumab, infliximab, ustekinumab and any combination thereof. Each possibility is a separate embodiment.

According to some embodiments, the methods further comprise the administration of a therapeutically effective amount of at least a second pharmaceutical agent, such as, but not limited to, a systemic drug or a Janus Kinase inhibitor (also referred to herein as “JAKi”).

According to some embodiments, the systemic drug may be selected from the group consisting of Methotrexate, Acitretin, Isotretinoin, Tegison, Cyclosporine, Apremilast, any pharmaceutically acceptable prodrug, metabolite, polymorph, salt, solvate (e.g., hydrate) or clathrate thereof and combinations thereof. Each possibility is a separate embodiment.

According to some embodiments, the JAKi is selected from the group consisting of tofacitinib, ruxolitinib, oclacitinib, baricitinib, filgotinib, gandotinib, lestaurtinib, momelotinib, pacritinib, upadacitinib (ABT-494), peficitinib, cucurbitacin I, CHZ868, fedratinib, cerdulatinib, ATI-50001, Leo-124429, or a salt or solvate thereof. Each possibility is a separate embodiment.

According to some embodiments, the at least one additional therapeutic agent may be administered together with the at least one biologic. According to some embodiments, the at least one additional therapeutic agent may be administered before or after (e.g. 1 week before/after or 1 month before/after) the administration of the at least one biologic.

According to some embodiments, the at least one additional therapeutic agent may enhance the influence of the biologic on the phototherapy (e.g. further increase its efficiency) and vice-versa. According to some embodiments, the at least one additional therapeutic agent may reduce/contradict the influence of the biologic on the phototherapy (e.g. reduce the increased efficiency caused by treatment of biologics and phototherapy and vice-versa.

According to some embodiments, determining the optimal dose of UVB may be influenced by it being administered in conjunction with a biologic and/or a systemic drug and/or a JAKi. According to some embodiments, the optimal dose of the biologic may be influenced by the coadministration of phototherapy and/or systemic drug and/or JAKi. According to some embodiments, the optimal dose of the systemic drug may be influenced by the coadministration with phototherapy and/or biological drug and/or JAKi. According to some embodiments, the optimal dose of the JAKi may be influenced by the coadministration with phototherapy and/or systemic drug and/or biological drug.

According to some embodiments, the JAKi is selected from the group consisting of tofacitinib, ruxolitinib, oclacitinib, baricitinib, filgotinib, gandotinib, lestaurtinib, momelotinib, pacritinib, upadacitinib (ABT-494), peficitinib, cucurbitacin J, CHZ868, fedratinib, cerdulatinib, ATI-50001, Leo-124429, or a salt or solvate thereof. Each possibility is a separate embodiment.

According to some embodiments, the JAKi is tofacitinib, or a salt or solvate thereof.

According to some embodiments, the JAKi is ruxolitinib, or a salt or solvate thereof.

As used herein and unless otherwise indicated, the term “pharmaceutically acceptable salt” includes, but is not limited to, salts prepared from pharmaceutically acceptable non-toxic acids or bases including inorganic acids and bases and organic acids and bases. Suitable pharmaceutically acceptable base addition salts provided herein include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts.

As used herein and unless otherwise indicated, the term “hydrate” means a compound provided herein or a salt thereof, that further includes a stoichiometric or non-stoichiometric amount of water bound by non-covalent intermolecular forces.

As used herein and unless otherwise indicated, the term “solvate” means a solvate formed from the association of one or more solvent molecules to a compound provided herein. The term “solvate” includes hydrates (e.g., mono-hydrate, dihydrate, trihydrate, tetrahydrate and the like).

As used herein and unless otherwise indicated, the term “polymorph” means solid crystalline forms of a compound provided herein or complex thereof. Different polymorphs of the same compound can exhibit different physical, chemical and/or spectroscopic properties.

As used herein and unless otherwise specified, the term “prodrug” means a derivative of a compound that can hydrolyze, oxidize, or otherwise react under biological conditions (in vitro or in vivo) to provide the compound. Examples of prodrugs include, but are not limited to, derivatives and metabolites of apremilast that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Prodrugs can typically be prepared using well-known methods, such as those described by 1 Burger's Medicinal Chemistry and Drug Discovery, 172-178, 949-982 (Manfred E. Wolff ed., 5th ed. 1995).

As used herein, and unless otherwise specified, the term “enantiomer,” “isomer” or “stereoisomer” encompasses all enantiomerically/stereomerically pure and enantiomerically/stereomerically enriched compounds provided herein.

As used herein, and unless otherwise indicated, the term “stereomerically pure” or “enantiomerically pure” means that a compound comprises one stereoisomer and is substantially free of its counter stereoisomer or enantiomer. For example, a compound is stereomerically or enantiomerically pure, when the compound contains greater than or equal to 80%, 90%, 95%, 96%, 97%, 98% or 99% of one stereoisomer, and 20%, 10%, 5%, 4%, 3%, 2%, 1% or less of the counter stereoisomer. “Substantially free of its (R) enantiomer” is encompassed by the term stereomerically pure or enantiomerically pure.

According to some embodiments, the biological drug may be administered in a composition providing sustained release. As used herein, the term “sustained release” refers to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material that provides sustained release properties to the compounds. As such, the compounds for use of the method of the invention may be administered in the form of microparticles, for example, by injection or in the form of wafers or discs by implantation.

According to some embodiments, the biological drug may be administered in a composition providing delayed release. As used herein, the term “delayed release” refers to a drug formulation that provides for an initial release of the drug after some delay following drug administration and that may, although not necessarily, include a delay of from about 10 minutes up to about 12 hours.

Dosing the therapeutically effective amount or dose of a compound of the present invention depends on the age, sex and weight of the patient, the current medical condition of the patient and the progression of a disease or disorder contemplated in the invention. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days.

In certain embodiments, the phototherapy is ultraviolet B (UVB, 280-320 nm) phototherapy. In other embodiments, the phototherapy is narrowband ultraviolet B (nbUVB, 311-312 nm) phototherapy. In yet other embodiments, the phototherapy is ultraviolet A (UVA, 320-340 nm) phototherapy. In yet other embodiments, the phototherapy is ultraviolet Al (UVA1, 340-400 nm) phototherapy. In yet other embodiments, the phototherapy is visible light (400-700 nm) phototherapy. In yet other embodiments, the phototherapy is a combination of phototherapies, including, but not limited to, those listed above.

According to some embodiments, the UVB light is UVB laser light. According to some embodiments, an excimer laser is used to generate the UVB laser light, although any other laser capable of emitting light in the UVB range can also be envisaged and, as such, is encompassed in the present disclosure. An excimer laser is a laser which uses a rare-gas halide or rare-gas metal vapor and emits laser light in the ultraviolet (126 to 558 nm) range. The laser used should operate in a range between 290 and 320 nm in wavelength, the UVB range of light. The laser should be utilized at a setting of not more than 120 mwatts. According to some embodiments, the laser is a 308 nm excimer laser.

As used herein, the term “conjunction” with regards to the biological drug, administered to the subject in conjunction with the phototherapy, refers to a treatment regimen including the administering of biological drug and phototherapy in such manner that at least one of the treatments effects the other, e.g. in such manner that the effect of the biological drug is enhanced by the phototherapy, in such manner that the biological drug affects the phototherapy (for example the tolerance to the phototherapy) and the like.

According to some embodiments, the treatments may be provided essentially simultaneously. As a non-limiting example, the biological drug may be administered shortly (e.g. 0-5 hours) before (or after) the phototherapy. According to some embodiments, the treatments may be provided sequentially. As a non-limiting example, the biological drug may be administered before or after (a day before, a day after, a week before or a week after) the phototherapy. According to some embodiments, the treatments may be provided sequentially during part of the regimen and simultaneously during other parts of the regimen. As a non-limiting example, the biological drug may initially be provided alone (e.g. for a week prior to the phototherapy) whereafter the treatments are provided essentially simultaneously (e.g. administration of the biological drug shortly before the phototherapy).

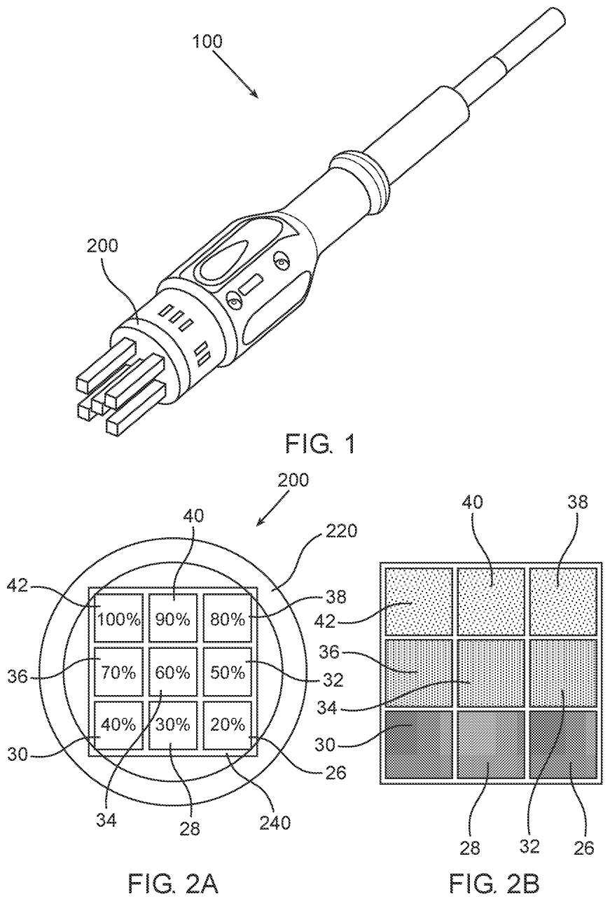

Reference is now made to FIG. 1, which illustratively depicts a hand-held phototherapy delivery apparatus 100 including a dosimetry device 200, configured to distribute a dose of light energy into a plurality of doses of varying levels of light energy that can then be applied onto a treatment area simultaneously or sequentially, to determine an optimum therapeutic dose of phototherapy for an individual suffering from a skin condition, by measuring the individual's treatment response, e.g. minimum blistering dose. By treating an individual suffering from a skin condition at or near their minimum blistering dose, the overall number of treatment sessions required to place the individual's diseased skin into remission can be greatly reduced, while burning of the individual's skin can be substantially reduced, and in most instances avoided.

As seen in FIG. 2A and FIG. 2B, dosimetry device 200 includes a housing 220 that is configured to be releasably connected to phototherapy delivery apparatus 100. Dosimetry device 200 includes a sensitivity matrix 240 arranged within housing 220. Housing 220 is here depicted as being cylindrical. However, any other shape, including, but not limited to, square, rectangular, elliptical, triangular, and trapezoidal are also envisaged and, as such, within the scope of this disclosure. Sensitivity matrix 240 can be connected (permanently or releasably) to housing 220 in any known manner.

Sensitivity matrix 240 is comprised of a plurality of regions 26, 28, 30, 32, 34, 36, 38, 40, 42 that are each designated to allow a prescribed intensity of light to pass therethrough and thus to assess an individual's maximum tolerated dose and, in turn, optimally to treat the patient at their maximum tolerable dose. Sensitivity matrix 240 is here depicted to include nine regions. However, matrix 240 can be comprised of any number of regions that can be arranged in any desired pattern to change what would have otherwise been a single unique dose level into an array of multiple dose levels simultaneously covering the range of potentially applicable therapeutic treatment levels.

According to some embodiments, regions 26, 28, 30, 32, 34, 36, 38, 40, 42 of sensitivity matrix 240 are comprised of absorptive and/or reflective material that allows for varying intensities of light to pass therethrough. In another embodiment, regions 26, 28, 30, 32, 34, 36, 38, 40, 42 of sensitivity matrix 240 are each comprised of partially transmissive material or filters that allows for varying intensities of light to pass therethrough.

According to some embodiments, sensitivity matrix 240 is comprised of fused silica optical components. According to some embodiments, regions 26, 28, 30, 32, 34, 36, 38, 40, 42 of sensitivity matrix 240 can be comprised of totally and/or partially reflective materials. The reflective materials can be a dielectric interference filter (e.g., partial reflector). According to some embodiments, the filter can be a multi-dielectric interference filter. According to some embodiments, the filter can be a metallic coating, including a dielectric enhanced metallic reflector. According to some embodiments, the filter can be metallic and comprised of materials such as aluminum or silver. In an embodiment, the filter can be a combination of dielectric interference filter, a multi-dielectric interference filter and a metallic coating.

According to some embodiments, the filters reflect a fraction of a dose of energy between about 0% and 99% and segment the dose into multiple beams or streams of energy of varying intensities and transmit the multiple beams or streams of energy of varying intensities onto an individual.

According to some embodiments, the intensity of light that is able to pass through regions 26, 28, 30, 32, 34, 36, 38, 40, 42 of sensitivity matrix 240 can range from approximately about 20% to 100%. According to some embodiments, the intensity of light that is able to pass through regions 26, 28, 30, 32, 34, 36, 38, 40, 42 of sensitivity matrix 240 can range from approximately about 20% to 90%. However, the number, shape and intensity of light being permissible to pass through the region 26, 28, 30, 32, 34, 36, 38, 40, 42 of sensitivity matrix 240 can vary and be greater or smaller than the numbers described herein.

The method for providing localized treatment of a skin condition is further elaborated on in the below described flowcharts. The flow charts are, for clarity reasons, described as separate embodiments. However, a person of ordinary skill in the art may understand that steps of one method may be incorporated into or substitute a step of another method, and such incorporation/substitution is thus a part of the present disclosure. It is further understood that whereas some steps are obviously sequential, the order of others may be changed and/or be performed simultaneously.

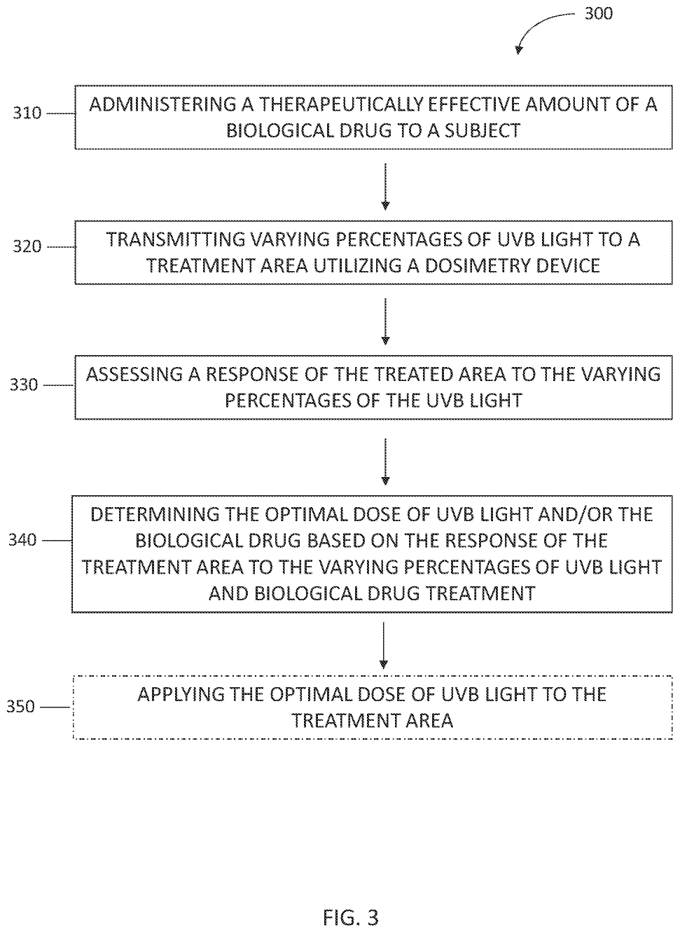

Reference is now made to FIG. 3, which is flowchart of a method 300 for localized treatment of a skin condition; according to some embodiments.

In step 310 of the method a therapeutically effective amount of a biological is administered to the subject. According to some embodiments, the biological is selected from the group consisting of: alefacept, etanercept, adalimumab, infliximab, ustekinumab and any combination thereof. Each possibility is a separate embodiment.

Following administration (e.g. about one hour after, about a day after, or about a week after administration of the biological), the subject's response/tolerance level of UVB light may be determined by transmitting various percentages of UVB light to the area of the subject's skin afflicted with the skin condition, using a dosimetry device, such as dosimetry device 200 of FIG. 2 (steps 320 and 330). Based on the response/tolerance level of UVB light, an optimal dose (optionally the maximal tolerated dose) of UVB light may be determined (step 340). According to some embodiments, the response/tolerance level of UVB light and thus the optimal dose, may be affected by the administration of the biological.

The optimal dose of UVB light determined in step 340 may then be applied to the subject (step 350).

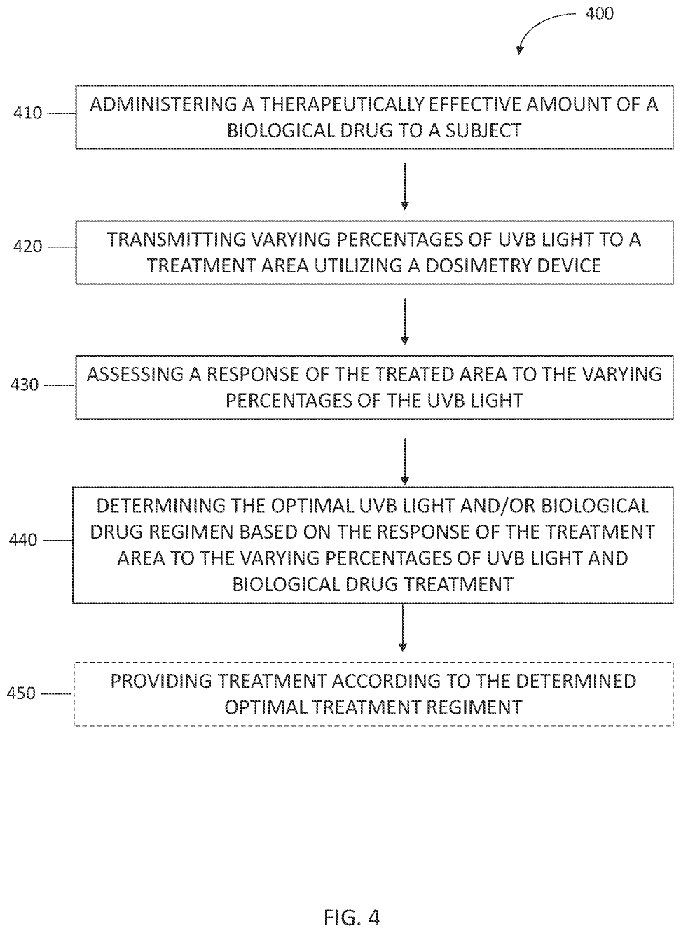

Reference is now made to FIG. 4, which is flowchart of a method 400 for localized treatment of a skin condition; according to some embodiments.

In step 410 of the method, a therapeutically effective amount of a biological is administered to the subject. According to some embodiments, the biological is selected from the group consisting of: alefacept, etanercept, adalimumab, infliximab, ustekinumab and any combination thereof. Each possibility is a separate embodiment.

Following administration (e.g. about one hour after, about a day after, or about a week after administration of the biological), the subject's response/tolerance level of UVB light may be determined by transmitting various percentages of UVB light to the area of the subject's skin afflicted with the skin condition, using a dosimetry device, such as dosimetry device 200 of FIG. 2 (steps 420 and 430). Based on the response/tolerance level of UVB light an optimal dose (optionally the maximal tolerated dose) of UVB light may be determined (step 440). According to some embodiments, the response/tolerance level of UVB light and thus the optimal dose, may be affected by the administration of the biological. According to some embodiments, the optimal dose of UVB light determined may also affect the optimal dose of the biological (which may need to be adjusted accordingly) as well as the regiment of the combined treatment (e.g. frequency of treatment, intensity of treatment, sequence of treatment, interval between treatments, etc.). Accordingly, the method may include a step 440 of determining the optimal UVB light and/or biological treatment regimen, based on the response of the treatment area to the varying percentages of UVB light and the biological treatment. According to some embodiments, the method may further include a step 450 of providing treatment according to the determined optimal treatment regimen.

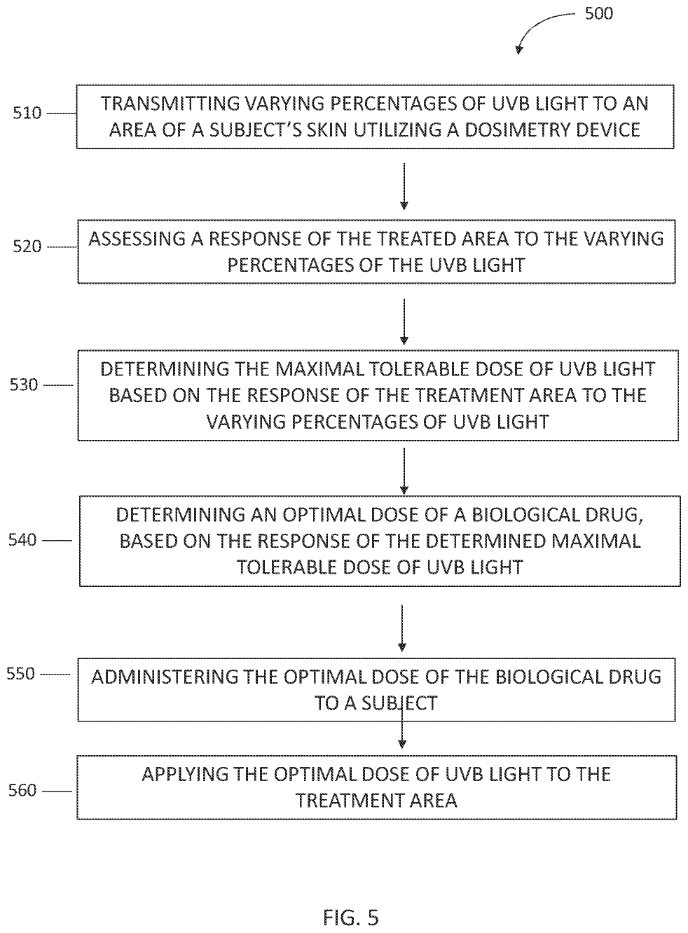

Reference is now made to FIG. 5, which is flowchart of a method 500 for localized treatment of a skin condition; according to some embodiments.

Initially, the method a subject's response/tolerance level to UVB light may be determined by transmitting various percentages of UVB light to an area of the subject's skin afflicted with the skin condition, using a dosimetry device, such as dosimetry device 200 of FIG. 2 (steps 510 and 520). Based on the response/tolerance level of UVB light, a maximal tolerated dose of UVB light may be determined (step 530). The optimal dose of the biological may then be determined according to the maximal tolerated dose of UVB light (steps 540). According to some embodiments, the biological is selected from the group consisting of: alefacept, etanercept, adalimumab, infliximab, ustekinumab and any combination thereof. Each possibility is a separate embodiment. Without being bound by any theory, when only low levels of UVB light are tolerated, higher doses of the biological drug may be needed to obtain an optimal effect. If, on the other hand, large doses of UVB are tolerated, a lower dose of the biological drug may be required, thus reducing both cost and potential side effects of the treatment. Once, the optimal dose of biological drug is determined, the subject may be administered therewith (step 550) followed by a UVB treatment, as set forth in step 560.

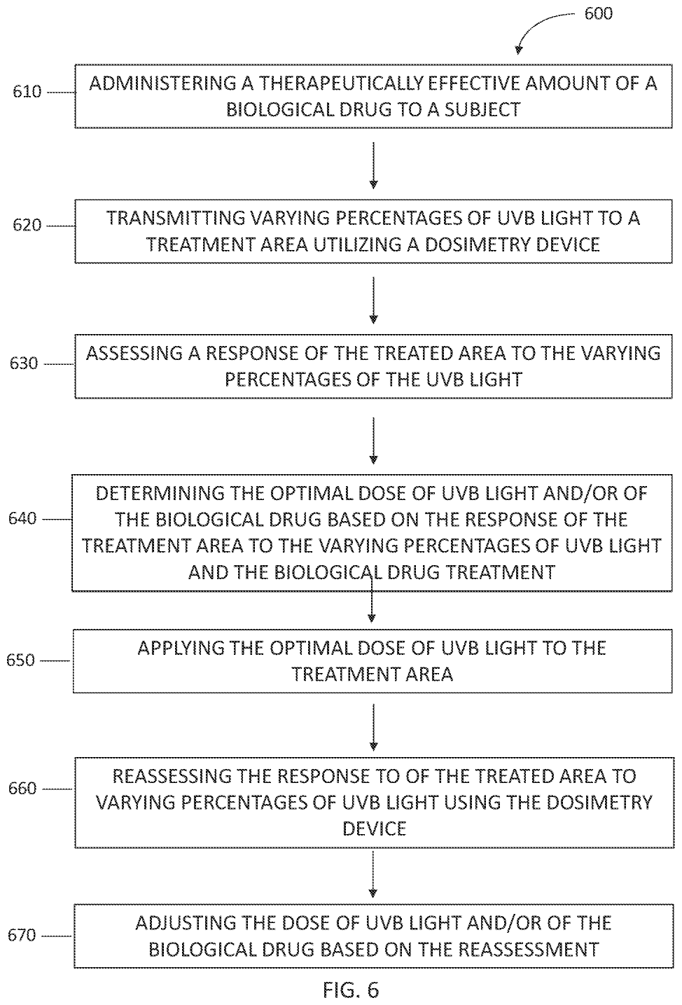

Reference is now made to FIG. 6, which is flowchart of a method 600 for localized treatment of a skin condition; according to some embodiments.

In step 610 of the method, a therapeutically effective amount of a biological is administered to the subject. According to some embodiments, the biological is selected from the group consisting of: alefacept, etanercept, adalimumab, infliximab, ustekinumab and any combination thereof. Each possibility is a separate embodiment.

Following administration (e.g. about one hour after, about a day after, or about a week after administration of the biological), the subject's response/tolerance level of UVB light may be determined by transmitting various percentages of UVB light to the area of the subject's skin afflicted with the skin condition, using a dosimetry device, such as dosimetry device 200 of FIG. 2 (steps 620 and 330). Based on the response/tolerance level of UVB light, an optimal dose (optionally the maximal tolerated dose) of UVB light may be determined (steps 640 and 650). According to some embodiments, the response/tolerance level of UVB light and thus the optimal dose, may be affected by the administration of biological and vice versa. Accordingly, the method may include an additional step of reassessing the subject's response to different doses of UVB light using the dosimetry device, as described (step 660) and adjusting the dose of UVB light and/or biological administered, based on the reassessment (step 670).

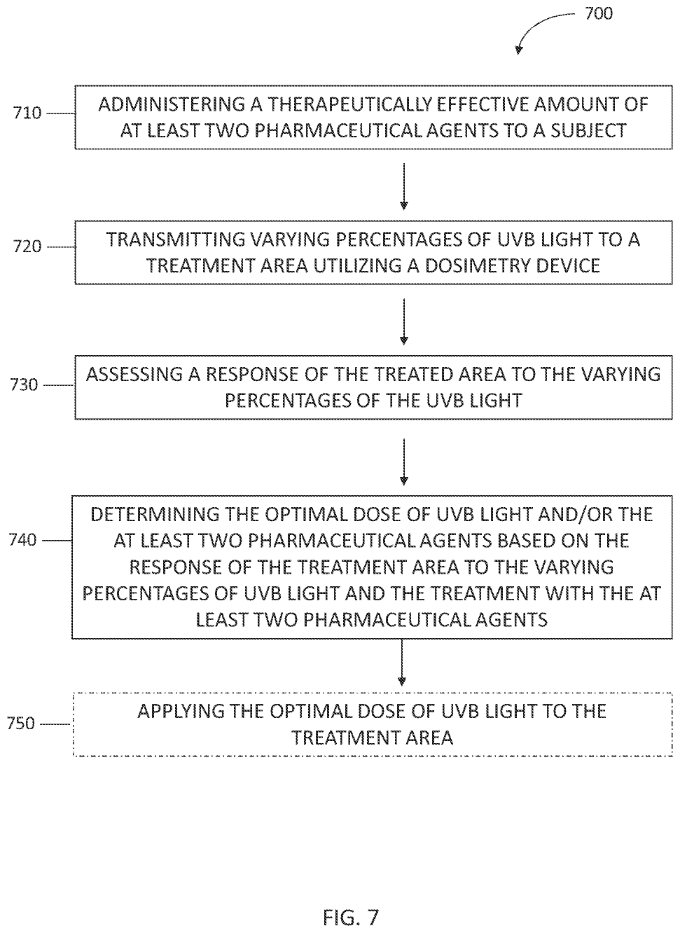

Reference is now made to FIG. 7, which is flowchart of a method 700 for localized treatment of a skin condition; according to some embodiments.

In step 710 of the method, a therapeutically effective amount of at least two pharmaceutical agents is administered to the subject.

According to some embodiments, the at least two pharmaceutical agents comprise a biological drug, a systemic drug or a JAKi. Each possibility is a separate embodiment.

According to some embodiments, the biological is selected from the group consisting of: alefacept, etanercept, adalimumab, infliximab, ustekinumab and any combination thereof. Each possibility is a separate embodiment.

According to some embodiments, the systemic drug is selected from the group consisting of: Methotrexate, Acitretin, Isotretinoin, Tegison, Cyclosporine, Apremilast, any pharmaceutically acceptable prodrug, metabolite, polymorph, salt, solvate (e.g., hydrate) or clathrate thereof and combinations thereof.

According to some embodiments, the JAKi is selected from the group consisting of tofacitinib, ruxolitinib, oclacitinib, baricitinib, filgotinib, gandotinib, lestaurtinib, momelotinib, pacritinib, upadacitinib (ABT-494), peficitinib, cucurbitacin I, CHZ868, fedratinib, cerdulatinib, ATI-50001, Leo-124429, or a salt or solvate thereof. Each possibility is a separate embodiment.

Following administration (e.g. about one hour after, about a day after, or about a week after administration of the at least two pharmaceutical agents), the subject's response/tolerance level of UVB light may be determined by transmitting various percentages of UVB light to the area of the subject's skin afflicted with the skin condition, using a dosimetry device, such as dosimetry device 200 of FIG. 2 (steps 720 and 730). Based on the response/tolerance level of UVB light, an optimal dose (optionally the maximal tolerated dose) of UVB light may be determined (step 740). According to some embodiments, the response/tolerance level of UVB light and thus the optimal dose, may be affected by the administration of the at least two pharmaceutical agents. Once the optimal dose has been determined in step 740, it may optionally be applied in step 750.

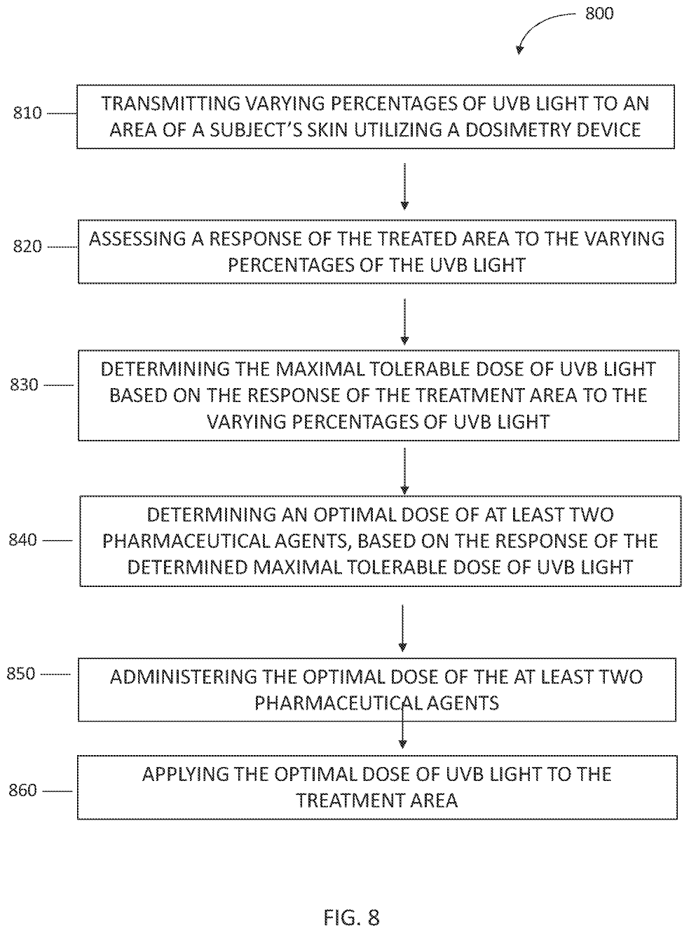

Reference is now made to FIG. 8, which is flowchart of a method 800 for localized treatment of a skin condition; according to some embodiments.

Initially, the method a subject's response/tolerance level to UVB light may be determined by transmitting various percentages of UVB light to an area of the subject's skin afflicted with the skin condition, using a dosimetry device, such as dosimetry device 200 of FIG. 2 (steps 810 and 820). Based on the response/tolerance level of UVB light, a maximal tolerated dose of UVB light may be determined (step 830). The optimal dose of the at least two pharmaceutical agents may then be determined according to the maximal tolerated dose of UVB light (steps 840 and 850).

According to some embodiments, the at least two pharmaceutical agents comprise a biological drug, a systemic drug or a JAKi

According to some embodiments, the biological is selected from the group consisting of: alefacept, etanercept, adalimumab, infliximab, ustekinumab and any combination thereof. Each possibility is a separate embodiment.

According to some embodiments, the systemic drug is selected from the group consisting of: Methotrexate, Acitretin, Isotretinoin, Tegison, Cyclosporine, Apremilast, any pharmaceutically acceptable prodrug, metabolite, polymorph, salt, solvate (e.g., hydrate) or clathrate thereof and combinations thereof.

According to some embodiments, the JAKi is selected from the group consisting of tofacitinib, ruxolitinib, oclacitinib, baricitinib, filgotinib, gandotinib, lestaurtinib, momelotinib, pacritinib, upadacitinib (ABT-494), peficitinib, cucurbitacin I, CHZ868, fedratinib, cerdulatinib, ATI-50001, Leo-124429, or a salt or solvate thereof. Each possibility is a separate embodiment.

Without being bound by any theory, when only low levels of UVB light are tolerated, higher doses of the at least two pharmaceutical agents may be needed to obtain an optimal effect. If, on the other hand, large doses of UVB are tolerated, a lower dose of the at least two pharmaceutical agents may potentially be required, thus reducing both cost and potential side effects of the treatment. Once, the optimal dose of the at least two pharmaceutical agents is determined, the subject may be administered therewith followed by a UVB treatment as set forth in step 860.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

1.-34. (canceled)

35. A method for localized treatment of a skin condition, the method comprising the steps of:
a. administering a therapeutically effective amount of at least one biological drug to the subject;
b. utilizing a dosimetry device, comprising an optical matrix comprising a plurality of regions, each region configured to allow varying percentages of UVB light to pass therethrough, to transmit varying percentages of the UVB light to an area of the subject's skin;
c. assessing a response of the treated area to the varying percentages of the UVB light transmitted thereto;
d. determine an optimal dose of UVB light, based on the response of the treated area to the varying percentages of UVB light and the biological drug administered; and
e. applying the optimal dose of UVB light to the treatment area.

36. The method of claim 35, wherein the optimal dose of UVB light is the maximum tolerable dose of UVB light and wherein the method further comprises determining an optimal amount of the biological drug based on the determined maximum tolerable dose of UVB light.

37. The method of claim 35, wherein the transmission of light passing through the regions ranges from about 20% in one region up to about 100% in another region.

38. The method of claim 35, wherein the UVB light is UVB laser light having a wavelength of about 290-320 nm.

39. The method of claim 35, wherein the UVB light is UVB laser light having a wavelength of about 308 nm and wherein the UVB laser light has an intensity of 60 mwatts.

40. The method of claim 35, wherein the administering of the biological drug and the applying of the maximum tolerable dose of UVB light is repeated 1-5 times a week.

41. The method of claim 35, wherein the administering of the therapeutically effective amount of the at least one biological drug is initiated at least 2 days prior to the transmitting of the varying percentages of UVB light to a treatment area and the assessment of the response of the treated area to the varying percentages of the UVB light transmitted thereto.

42. The method of claim 35, wherein the at least one biological drug is selected from the group consisting of: alefacept, etanercept, adalimumab, infliximab, ustekinumab and any combination thereof.

43. The method of claim 35, wherein the skin condition is selected from vitiligo, psoriasis, leukoderma, atopic dermatitis, dyshidrosis, eczema, alopecia areata and lichen planus.

44. The method of claim 43, wherein the skin condition is vitiligo or psoriasis.

45. A method for localized treatment of a skin condition, the method comprising the steps of:
a. utilizing a dosimetry device, comprising an optical matrix comprising a plurality of regions, each region configured to allow varying percentages of UVB light to pass therethrough, to transmit varying percentages of the UVB light to an area of the subject's skin affected with the skin condition;
b. assessing a response of the skin area to the varying percentages of the UVB light transmitted thereto;
c. determining an optimal dose of UVB light, based on the response of the treated skin area to the varying percentages of UVB light;
d. determining an optimal dose of at least one biological drug, based on the determined optimal dose of UVB light;
e. administering the optimal dose of the biological drug to the subject,
f. treating the skin area with the optimal dose of UVB light.

46. The method of claim 45, wherein the optimal dose of UVB light is the maximum tolerable dose of UVB light.

47. The method of claim 46, wherein the transmission of light passing through the regions ranges from about 20% in one region up to about 100% in another region.

48. The method of claim 45, wherein the UVB light is UVB laser light having a wavelength of about 290-320 nm.

49. The method of claim 48, wherein the UVB light is UVB laser light having a wavelength of about 308 nm and an intensity of 60 mwatts.

50. The method of claim 45, wherein the administering of the biological drug and the applying of the maximum tolerable dose of UVB light is repeated 1-5 times a week.

51. The method of claim 45, wherein the at least one biological drug is selected from the group consisting of: alefacept, etanercept, adalimumab, infliximab, ustekinumab and any combination thereof.

52. The method of claim 45, wherein the skin condition is selected from vitiligo, psoriasis, leukoderma, atopic dermatitis, dyshidrosis, eczema, alopecia areata and lichen planus.

53. The method of claim 52, wherein the skin condition is vitiligo or psoriasis.
